(19) United States
(12) Patent Application Publication
(10) Pub. No.: US 2023/0086247 A1
(43) Pub. Date: Mar. 23, 2023

(54) System and Method for Separating and In-Situ Analyzing A Multiphase Immiscible Fluid Mixture

(71) Applicant: Saudi Arabian Oil Company, Dhahran (SA)

(72) Inventors: Naim Akmal, Dhahran (SA); Ahmed Khalid Alqatari, Dhahran (SA)

(73) Assignee: Saudi Arabian Oil Company, Dhahran (SA)

(21) Appl. No.: 17/482,098

(22) Filed: Sep. 22, 2021

(51) Int. Cl. C02F 1/469; G01N 1/10; G01N 33/18; G01N 1/38

(52) U.S. Cl. CPC C02F 1/4693; G01N 1/10; G01N 33/18; G01N 1/38; C02F 2201/46115; C02F 2209/10; C02F 2209/06; C02F 2209/19; C02F 2209/15

(57) ABSTRACT

A system separates and in-situ analyzes a discrete sample of multiphase fluid. The system includes a separation vessel having a first inner chamber for separating a discrete sample of multiphase fluid into liquid phases including an aqueous liquid phase and a nonporous liquid phase, and a built-in water analysis unit. The built-in water analysis unit includes an analytical cell disposed inside the first inner chamber of the separation vessel, the analytical cell having a second inner chamber, and at least one probe having a sensing area disposed in the second inner chamber for in-situ analysis of a sample of the aqueous liquid phase that is separated from the discrete sample of multiphase fluid in the first inner chamber and that is channeled to the second inner chamber from the first inner chamber for the in-situ analysis. The second inner chamber is defined inside the first inner chamber.

### TECHNICAL FIELD

Embodiments of the present disclosure relate generally to a system and method for separating and analyzing multiphase immiscible fluid mixture samples. More specifically, embodiments of the present disclosure relate to analyzing the separated multiphase immiscible fluid mixture sample in-situ, with a water analysis unit built-in inside a separation vessel.

### BACKGROUND

Multiphase immiscible fluid mixtures (e.g., multiphase fluids) produced from oil wells typically are a mixture of gas, liquid hydrocarbons, and salty formation water (e.g., produced water). For example, an oil well may produce polar and nonpolar molecules along with gases such as carbon dioxide, hydrogen sulfide, carbon disulfide, and the like. A gas oil separation plant (GOSP) is used in the upstream oil and gas industry to refer to temporary or permanent facilities that separate the multiphase fluids obtained from a plurality of wells (e.g., more than a hundred oil wells) into constituent vapor and liquid components (e.g., liquid hydrocarbons, and salty formation or produced water) and generate dry crude oil that meets predetermined customer specifications. A typical GOSP includes a high-pressure production trap (HPPT), a low pressure production trap (LPPT), a low pressure degassing tank (LPDT), a dehydrator unit, first and second stage desalting units, a water/oil separation plant (WOSEP), a stabilizer column, centrifugal pumps, heat exchangers, and reboilers.

Composition of the multiphase fluid produced from each well feeding into the GOSP typically varies over time. Generally, a greater amount of crude oil is produced initially from the well. Over time, the amount of produced water increases and the amount of crude oil produced decreases. It is necessary to know the amount of crude oil (and produced water) produced from each well of the GOSP in order to manage production of each well, while maintaining overall efficiency of the GOSP and generating dry crude that meets customer specifications. For example, if a particular well is producing a high proportion of water, it may be desirable to isolate the well from the flow of the GOSP.

A multiphase flow meter (MPFM) may be used at the GOSP (or at a well site upstream the GOSP) to measure the amount or rate of crude oil (and produced water) produced from each well. The MPFM's built-in software and algorithm can be utilized to determine the flow of oil from the combined flow of produced water and crude oil. To obtain accurate measurement of the amount or flow rate of crude oil passing through the MPFM, it is necessary to calibrate the MPFM using predetermined data representing certain physical or chemical properties of the produced water contained in the multiphase fluid (including oil and water) passing through the MPFM. That is, it is necessary to enter data regarding certain properties of the produced water into the MPFM panel so that the flow meter displays information regarding the flow of the constituent oil of the multiphase fluid with high accuracy. To perform such calibration, conventionally, a sample of the multiphase fluid (from one well or a group of wells whose output is passing through the MPFM) is periodically collected in a test trap. The test trap can be rated as having high pressure, intermediate pressure, or low pressure. Crude oil in the sample is allowed to separate from produced water in the test trap, and a portion of the separated produced water is collected and sent to a local laboratory to analyze certain geophysical or geochemical properties (e.g., salinity, chloride content, conductivity, and the like) of the separated produced water sample. The data obtained by this analysis is used to calibrate the MPFM. More specifically, the analytical result received from the laboratory is manually fed into the MPFM panel to optimize or calibrate the output of the MPFM (i.e., optimize oil flow rate data and water flow rate data coming out of the MPFM).

The periodic act of collection of the separated produced water sample from the test trap, transferring the sample to the laboratory, measuring the geophysical properties of the sample in the laboratory, bringing the analytical data back to the GOSP, and manually feeding the analytical data into the MPFM, can take approximately two to three days. Further, since the analytical data received from the laboratory is manually fed into the MPFM, there is a possibility of introducing a human data entry error. A better approach that is faster, automated, low-maintenance, and less prone to human error is desirable.

### SUMMARY

The following presents a simplified summary of the disclosed subject matter in order to provide a basic understanding of some aspects of the subject matter disclosed herein. This summary is not an exhaustive overview of the technology disclosed herein. It is not intended to identify key or critical elements of the disclosed subject matter or to delineate the scope of the disclosed subject matter. Its sole purpose is to present some concepts in a simplified form as a prelude to the more detailed description that is discussed later.

In one embodiment, a system for separating and in-situ analyzing a discrete sample of multiphase fluid includes: a separation vessel having a first inner chamber for separating a discrete sample of multiphase fluid into liquid phases including an aqueous liquid phase and a nonporous liquid phase; and a built-in water analysis unit including: an analytical cell disposed inside the first inner chamber of the separation vessel, the analytical cell having a second inner chamber; and at least one probe having a sensing area disposed in the second inner chamber for in-situ analysis of a sample of the aqueous liquid phase that is separated from the discrete sample of multiphase fluid in the first inner chamber and that is channeled to the second inner chamber from the first inner chamber for the in-situ analysis, where the second inner chamber is defined inside the first inner chamber. In another embodiment, the at least one probe has an oblong shape, and wherein the sensing area of the probe is covered with an ion-exchange membrane to prevent fouling of the sensing area.

In yet another embodiment, the analytical cell is built-in in a bottom portion of the separation vessel such that an opening of the sample control valve is disposed in a bottom region of the first inner chamber, where the aqueous liquid phase is likely to accumulate after separating from the discrete sample of multiphase fluid. In yet another embodiment, the analytical cell has a sample inlet and wherein the second inner chamber is in fluid communication with the first inner chamber via the sample inlet. In yet another embodiment, the built-in water analysis unit further includes a sample control valve coupled to the sample inlet for controlling a flow of the separate aqueous liquid phase from the first inner chamber to the second inner chamber, where the analytical cell further has a fresh water inlet, and the second inner chamber is in fluid communication with a fresh water reservoir via the fresh water inlet, and where the system further includes one or more processors operatively coupled to the sample control valve and the at least one probe, the one or more processors being configured to: control the sample control valve to channel a predetermined amount of the separate aqueous liquid phase as the aqueous liquid phase sample from the first inner chamber to the second inner chamber via the sample inlet; dilute the aqueous liquid phase sample channeled into the second inner chamber with a predetermined amount of fresh water introduced into the second inner chamber via the fresh water inlet, to generate a diluted aqueous liquid phase sample; in-situ analyze the diluted aqueous liquid phase sample in the second inner chamber with the at least one probe to obtain diluted aqueous liquid phase sample data; calculate nondiluted aqueous liquid phase sample data based on the diluted aqueous liquid phase sample data, as well as based on the predetermined amount of fresh water in the diluted aqueous liquid phase sample; and transmit the nondiluted aqueous liquid phase sample data to a multiphase flow meter for calibration.

In yet another embodiment, the sensing area of the at least one probe is at a distal end of the probe, and wherein the probe is oriented in the second inner chamber such that the sensing area is immersed in the diluted aqueous liquid phase sample when the diluted aqueous liquid phase sample is contained in the second inner chamber.

In yet another embodiment, the at least one probe includes an ion-selective electrode configured to in-situ measure one or more properties of the diluted aqueous liquid phase sample, the one or more properties selected from a group including: sodium concentration, chloride concentration, total dissolved solids (TDS) concentration, pH, conductivity, sulfate concentration, carbonate concentration, and nitrate concentration.

In yet another embodiment, the at least one probe includes first, second, and third probes that are proximally disposed adjacent to each other such that each probe is oriented in the second inner chamber with the sensing area of the probe in a downward direction and immersed in the diluted aqueous liquid phase sample when the diluted aqueous liquid phase sample is contained in the second inner chamber, and such that there exists an acute angle measured from the probe to a horizontal plane that is substantially perpendicular to a direction of gravity. In yet another embodiment, the acute angle is in the range of 30°-60°. In yet another embodiment, the one or more processors are further configured to: introduce the discrete sample of multiphase fluid into the first inner chamber of the separation vessel via a multiphase fluid inlet of the separation vessel; mix a predetermined amount of demulsifier obtained from a demulsifier source with the discrete sample of multiphase fluid in the first inner chamber to cause the discrete sample to separate into liquid phases including the aqueous liquid phase and the nonpolar liquid phase; and control the sample control valve to channel the predetermined amount of the aqueous liquid phase as the aqueous liquid phase sample from the first inner chamber to the second inner chamber via the sample inlet of the analytical cell, in response to determining that the discrete sample of multiphase fluid in the first inner chamber has separated into liquid phases including the aqueous liquid phase and the nonpolar liquid phase.

In yet another embodiment, the analytical cell further has a sample outlet, wherein the separation vessel has a drain outlet, and wherein the one or more processors are further configured to: drain the diluted aqueous liquid phase sample in the second inner chamber via the sample outlet after obtaining the diluted aqueous liquid phase sample data; rinse the second inner chamber and the sensing area of the at least one probe disposed in the second inner chamber with fresh water introduced into the second inner chamber via the fresh water inlet after draining the diluted aqueous liquid phase sample; and drain the discrete sample of multiphase fluid in the first inner chamber via the drain outlet after channeling the predetermined amount of the aqueous liquid phase as the aqueous liquid phase sample from the first inner chamber to the second inner chamber.

In yet another embodiment, the predetermined amount of the aqueous liquid phase channeled as the aqueous liquid phase sample from the first inner chamber to the second inner chamber is substantially in the range of 50-60 milliliters.

In yet another embodiment, a method for separating and in-situ analyzing a discrete sample of multiphase fluid includes: introducing a discrete sample of multiphase fluid into a first inner chamber of a separation vessel, wherein an analytical cell having a second inner chamber is built-in inside the first inner chamber of the separation vessel, and wherein the analytical cell has a sample inlet for fluidly communicating the second inner chamber with the first inner chamber; mixing a predetermined amount of demulsifier obtained from a demulsifier source with the discrete sample of multiphase fluid in the first inner chamber to cause the discrete sample to separate into liquid phases including an aqueous liquid phase and a nonpolar liquid phase; channeling a predetermined amount of the separate aqueous liquid phase as an aqueous liquid phase sample from the first inner chamber to the second inner chamber via the sample inlet of the analytical cell, in response to determining that the discrete sample of multiphase fluid in the first inner chamber has separated into liquid phases including the aqueous liquid phase and the nonpolar liquid phase; diluting the aqueous liquid phase sample channeled into the second inner chamber with a predetermined amount of fresh water from a fresh water reservoir to generate a diluted aqueous liquid phase sample; and in-situ analyzing the diluted aqueous liquid phase sample contained in the second inner chamber with at least one probe having a sensing area disposed in the second inner chamber, where the second inner chamber is defined inside the first inner chamber.

In yet another embodiment, the method further includes: obtaining diluted aqueous liquid phase sample data based on the in-situ analysis with the at least one probe; calculating nondiluted aqueous liquid phase sample data based on the diluted aqueous liquid phase sample data, as well as based on the predetermined amount of fresh water in the diluted aqueous liquid phase sample; and transmitting the nondiluted aqueous liquid phase sample data to a multiphase flow meter. In yet another embodiment, the analytical cell further has a sample outlet on a bottom surface thereof, wherein the separation vessel has a drain outlet on a bottom surface thereof, and where the method further includes: draining the diluted aqueous liquid phase sample in the second inner chamber via the sample outlet after obtaining the diluted aqueous liquid phase sample data; rinsing the second inner chamber and the sensing area of the at least one probe disposed in the second inner chamber with fresh water from the fresh water reservoir after draining the diluted aqueous liquid phase sample; and draining the discrete sample of multiphase fluid in the first inner chamber via the drain outlet after channeling the predetermined amount of the aqueous liquid phase as the aqueous liquid phase sample from the first inner chamber to the second inner chamber.

In yet another embodiment, a water analysis unit of a system for separating and in-situ analyzing a discrete sample of multiphase fluid includes: an analytical cell disposed inside a first inner chamber of a separation vessel for separating a discrete sample of multiphase fluid into liquid phases including an aqueous liquid phase and a nonporous liquid phase, wherein the analytical cell has: (i) a second inner chamber that is defined inside the first inner chamber, and (ii) a sample inlet to fluidly communicate the second inner chamber with the first inner chamber; and at least one probe having a sensing area disposed in the second inner chamber for in-situ analysis of a sample of the aqueous liquid phase that is separated from the discrete sample of multiphase fluid in the first inner chamber and that is channeled to the second inner chamber from the first inner chamber for the in-situ analysis.

In yet another embodiment, the at least one probe has an oblong shape, and wherein the sensing area of the probe is covered with an ion-exchange membrane to prevent fouling of the sensing area, where the analytical cell is built-in in a bottom portion of the separation vessel, and where an opening of the sample control valve is adapted to be disposed in a region of the first inner chamber where the aqueous liquid phase accumulates after separation thereof the discrete sample of multiphase fluid. In yet another embodiment, the analytical cell further has a fresh water inlet, and the second inner chamber is in fluid communication with an external fresh water reservoir via the fresh water inlet, and where the water analysis unit further includes: a sample control valve coupled to the sample inlet for controlling a flow of the aqueous liquid phase sample from the first inner chamber to the second inner chamber; and one or more processors operatively coupled to the sample control valve and the at least one probe, the one or more processors being configured to: control the sample control valve to allow a predetermined amount of the separate aqueous liquid phase to flow into the second inner chamber via the sample inlet as the aqueous liquid phase sample; dilute the aqueous liquid phase sample in the second inner chamber to generate a diluted aqueous liquid phase sample by allowing a predetermined amount of fresh water from the fresh water reservoir to flow into the second inner chamber via the fresh water inlet; in-situ analyze the diluted aqueous liquid phase sample in the second inner chamber with the at least one probe to obtain diluted aqueous liquid phase sample data; calculate nondiluted aqueous liquid phase sample data based on the diluted aqueous liquid phase sample data, and based on the predetermined amount of fresh water in the diluted aqueous liquid phase sample; transmit the nondiluted aqueous liquid phase sample data to an external multiphase flow meter.

In yet another embodiment, the sensing area of the at least one probe is at a distal end of the probe, and wherein the probe is oriented in the second inner chamber such that the sensing area is immersed in the diluted aqueous liquid phase sample when the diluted aqueous liquid phase sample is contained in the second inner chamber. In yet another embodiment, the at least one probe includes an ion-selective electrode configured to in-situ measure one or more properties of the diluted aqueous liquid phase sample, the one or more properties selected from a group including: sodium concentration, chloride concentration, total dissolved solids (TDS) concentration, pH, conductivity, sulfate concentration, carbonate concentration, and nitrate concentration. In yet another embodiment, the at least one probe includes first, second, and third probes that are proximally disposed adjacent to each other such that each probe is oriented in the second inner chamber with the sensing area of the probe in a downward direction and immersed in the diluted aqueous liquid phase sample when the diluted aqueous liquid phase sample is contained in the second inner chamber, and such that there exists an acute angle measured from the probe to a horizontal plane that is substantially perpendicular to a direction of gravity.

In yet another embodiment, the analytical cell further has a sample outlet on a bottom surface thereof, and the one or more processors are further configured to: drain the diluted aqueous liquid phase sample in the second inner chamber via the sample outlet after obtaining the diluted aqueous liquid phase sample data; and rinse the second inner chamber and the sensing area of the at least one probe disposed in the second inner chamber with fresh water from the fresh water reservoir after draining the diluted aqueous liquid phase sample. In yet another embodiment, the predetermined amount of the aqueous liquid phase allowed to flow into the second inner chamber via the sample inlet as the aqueous liquid phase sample is substantially in the range of 50-60 milliliters.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic illustration of a system for separating and in-situ analyzing an aqueous liquid phase sample separated from a discrete sample of multiphase fluid in a separation vessel, in accordance with one or more embodiments.

FIG. 2 is a flow chart that illustrates a method of operation of the system for separating and in-situ analyzing the aqueous liquid phase sample separated from the discrete sample of multiphase fluid in the separation vessel, in accordance with one or more embodiments.

FIG. 3 is a functional block diagram of an exemplary computer system, in accordance with one or more embodiments.

While certain embodiments will be described in connection with the illustrative embodiments shown herein, the subject matter of the present disclosure is not limited to those embodiments. On the contrary, all alternatives, modifications, and equivalents are included within the spirit and scope of the disclosed subject matter as defined by the claims. In the drawings, which are not to scale, the same reference numerals are used throughout the description and in the drawing figures for components and elements having the same structure.

### DETAILED DESCRIPTION

In the following description, for purposes of explanation, numerous specific details are set forth in order to provide a thorough understanding of the inventive concept. In the interest of clarity, not all features of an actual implementation are described. Moreover, the language used in this disclosure has been principally selected for readability and instructional purposes, and may not have been selected to delineate or circumscribe the inventive subject matter, resort to the claims being necessary to determine such inventive subject matter. Reference in this disclosure to “one embodiment” or to “an embodiment” or “another embodiment” means that a particular feature, structure, or characteristic described in connection with the embodiment is included in at least one embodiment of the disclosed subject matter, and multiple references to “one embodiment” or “an embodiment” or “another embodiment” should not be understood as necessarily all referring to the same embodiment.

This disclosure pertains to a system for separating and in-situ analyzing a sample of an aqueous liquid phase (e.g., produced water) separated from a discrete sample of multiphase fluid (e.g., oil-water mixture) in a separation vessel and corresponding method. The separated produced water sample is analyzed in a water analysis unit that is built-in inside an inner chamber of the separation vessel where the discrete sample of multiphase fluid has been separated into liquid phases including the aqueous liquid phase and a nonpolar liquid phase (e.g., oil). Since the water analysis unit is built-in inside the inner chamber of the separation vessel, a separate analytical cell or vessel external to the separation vessel for analysis and measurement of the separated produced water sample is not required, thereby reducing costs.

The built-in water analysis unit includes (e.g., contains, is equipped with, is disposed with, is installed with) one or more ion-selective electrodes (e.g., probes, sensors) to measure one or more properties (e.g., geophysical properties, geochemical properties, and the like) of the separated produced water sample that is channeled from an inner chamber of the separation vessel to an inner space of the analytical cell. For example, the measured properties include pH, conductivity, salinity, chloride content, sodium content, total dissolved solids (TDS), and other ions. More specifically, the water analysis unit built-in inside the inner chamber of the separation vessel includes an analytical cell that defines an inner space where one or more miniaturized ion-selective electrodes (e.g., sensors, probes, and the like) are disposed in series to measure the various properties of the produced water sample. For example, the ion-selective electrodes disposed in series in the inner space of the analytical cell include a first electrode to measure sodium concentration (for salinity), a second electrode to measure conductivity, and a third electrode to measure TDS concentration, and the system is configured to automatically and simultaneously operate the three electrodes, so that the three electrodes work together to measure predetermined properties of produced water sample. Having the water analysis unit built-in inside the inner chamber of the separation vessel enables continuous, real-time measurement of the properties of the separated produced water sample, without having to transfer the separated produced water sample from the separation vessel to an external analytical cell, thereby increasing efficiency and reducing costs.

Having an accurate view of the hydrocarbons produced from a well (at a GOSP or well site) enables operators to make better decisions regarding the economic potential of the well, and of the oil field more generally. Advantageously, the method and system with the built-in water analysis unit disclosed here are capable of providing near-instantaneous, real-time water sample measurements for multiphase fluid samples obtained from a well(s) that, when utilized to control, optimize or calibrate a MPFM, enables production engineers to obtain an accurate view regarding the hydrocarbon production of the well(s). For example, a well or group of wells producing a significant water cut can be identified, and isolated if necessary, so that resources are conserved. Because the system and method disclosed herein can be automated, measurements can be carried out routinely in an unattended and uninterrupted manner with minimal labor costs and reduced potential for error. More specifically, data obtained using the system and method disclosed here can be used to calibrate, optimize, or control the MPFM, so that accurate flow rates of each phase of the multiphase fluid flowing out of the well(s) can be measured over time. The measured data may also be used to assess the remaining productivity of the producing well(s). The system and method disclosed here thus enable real-time, faster, and more accurate measurement of data that provides the information necessary for the control and optimization of the oil field or of the GOSPs output.

In operation, a control unit of the system is configured to control flow of a multiphase fluid sample into the separation vessel with the built-in water analysis unit. The control unit may control to separate liquid phases (e.g., oil and produced water) of the multiphase fluid sample in the separation vessel by adding a predetermined measured amount (and/or type) of demulsifier to the multiphase fluid sample in the separation vessel and operating a mixer to actively mix the demulsifier into the multiphase fluid sample. Still further, the control unit may be configured to cause a measured amount of the produced water separated from the multiphase fluid sample to be introduced (channeled) into the analytical cell of built-in water analysis unit from the inner chamber of the separation vessel for in-situ measurement. The control unit may be configured to dilute the measured amount of the separated produced water contained in the built-in analytical cell with a measured amount of fresh water, and in-situ measure the geophysical or geochemical properties of the diluted produced water sample using one or more miniaturized sensors or probes (e.g., ion-selective electrodes) disposed inside the built-in analytical cell. The system is thus configured to perform the separation, analysis and measurement operations, inside the separation vessel, without the need to convey the separated produced water sample out of the separation vessel for analysis and measurement. The control unit may further be configured to transmit data representing the measured properties of the separated produced water sample to an already existing MPFM associated with one or more wells from which the multiphase fluid sample was obtained to calibrate, control, or optimize the flow rate measurements for each phase by the MPFM. The MPFM may thus continuously, quickly and automatically be calibrated using multiphase fluid samples obtained in real-time to continuously and accurately calculate the flow rate of the oil flowing from the GOSP (or oil field) at any given time.

The system and method of the present disclosure is thus capable of automatically monitoring geophysical or geochemical properties of produced water by taking continuous readings of multiphase fluid samples from one or more wells at the GOSP or oil field. The system can easily take samples and then measure in-situ the properties of the separated produced water for each sample and feed the measurement directly into the MPFM. The separation vessel with the built-in water analysis unit can be installed proximal to the MPFM, and the control unit can automatically divert samples from the well to the separation vessel with the built-in water analysis unit to analyze in-situ the geochemical properties of the produced water sample, and the control unit can further automatically transmit the measurement data for each sample from the built-in water analysis unit to the MPFM. Since the measurement data is automatically fed to the MPFM, manual sample collection and manual data entry into the MPFM is not required, and real-time measurement and monitoring for one or more wells at the GOSP or at the oil field can be automatically performed without requiring constant human supervision or interruption.

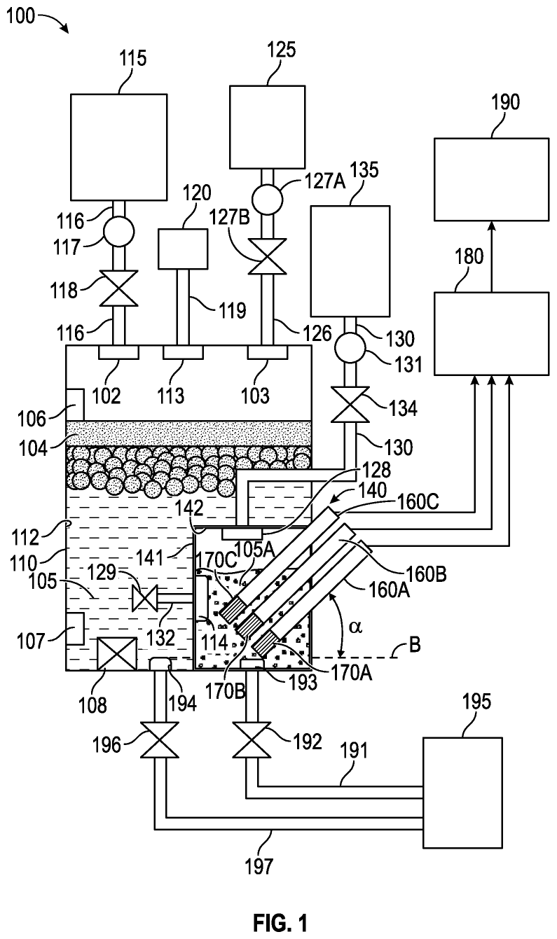

FIG. 1 is a schematic illustration of system 100 for separating and in-situ analyzing an aqueous liquid phase sample separated from a discrete sample of multiphase fluid, in accordance with one or more embodiments. System 100 represents the flow pattern and fill-up scheme of a multiphase fluid in the separation vessel, its separation, and real-time in-situ measurement of geophysical or geochemical properties of an aqueous liquid phase (e.g., produced water) separated from the multiphase fluid. As shown in FIG. 1, system 100 includes separation vessel (e.g., separation chamber) 110 defining inner chamber 112 (e.g., first inner chamber) where fluid can be stored. Separation vessel 110 may be manufactured from an at least partially translucent or transparent material such that the level of liquid inside vessel 110 can be determined by observation from outside separation vessel 110. For example, separation vessel 110 can be made of shatter-proof glass and can include markings for measuring the volume of liquid contained within.

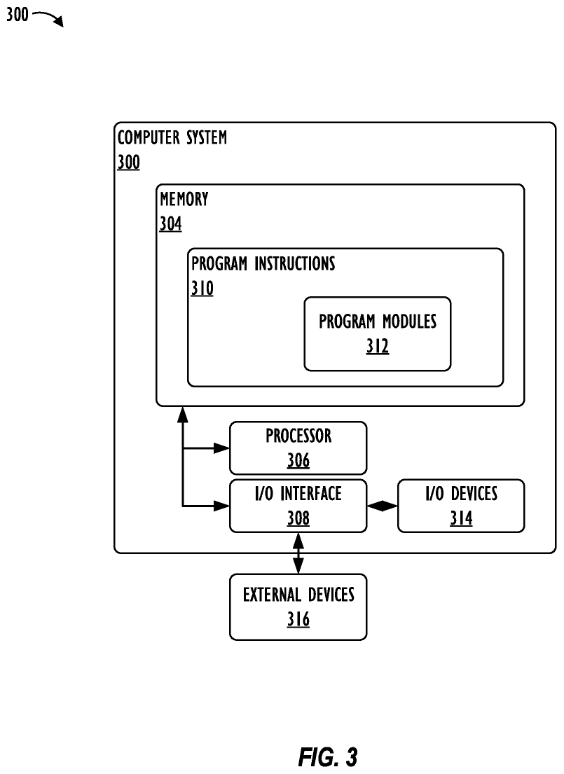

Separation vessel 110 may be configured to receive and contain a multiphase fluid from a selected well or group of wells associated with system 100. The well(s) may belong to an oil field that is serviced by a GOSP to separate the multiphase fluid produced from the well(s) into constituent vapor and liquid components, and generate dry crude oil. As shown in FIG. 1, system 100 further includes control unit 180 (e.g., programmable logic controller (PLC), central processing unit (CPU), graphics processing unit (GPU), system on a chip, application specific integrated circuit (ASIC), and the like) that may include predetermined control logic (implemented in hardware and/or software) and predetermined data to control and operate the various electronic components of system 100 shown in FIG. 1 to automate operations thereof. Although not specifically shown in FIG. 1, control unit 180 is communicatively coupled to the various electronic components of system 100 shown in FIG. 1 to communicate data and/or control signals therewith. Control unit 180 may be implemented on a computer system that is the same as or similar to computer system 300 described with regard to at least FIG. 3.

As shown in FIG. 1, separation vessel 110 has multiphase fluid inlet 102 in fluid communication with holding chamber 115 (e.g., holding tank, high-pressure fluid line, and the like) via multiphase fluid coupling 116 to receive a discrete sample of multiphase fluid, based on control operation of control unit 180. Holding chamber 115 may be a high pressure, intermediate pressure or low pressure test trap for the multiphase fluid from a selected source (e.g., from a well or group of wells). Alternately, in case system 100 is implemented at a GOSP, holding chamber 115 may correspond to a high-pressure sample line where the multiphase liquid from the selected source may be flowing at a high pressure.

Pump assembly 117 and inlet control valve 118 may be disposed (installed) on multiphase fluid coupling 116 to selectively start, stop, and control a flow rate of a stream of the multiphase fluid flowing through multiphase fluid coupling 116, based on control operations of control unit 180. Pump assembly 117 may be driven by one or more electric motors. Examples of electric motors used to drive pump assembly 117 include induction motors and/or permanent magnet motors. System 100 may further include one or more drives (e.g., variable frequency drives (VFDs); not shown) that monitor and control the electric motors, under control of control unit 180. The control drives, inlet control valve 118, and control unit 180 may together define a control system for automatically and selectively controlling (e.g., starting, stopping, changing flow rate, and the like) a flow of a measured amount of the multiphase fluid into separation vessel 110.

As shown in FIG. 1, separation vessel 110 may be equipped with first level indicator 106 (e.g., level sensor) and second level indicator 107 (e.g., level sensor). First and second level indicators 106 and 107 may be configured to detect a liquid level or fill level inside inner chamber 112 of separation vessel 110. For example, second level indicator 107 may detect when separation vessel 110 is empty (e.g., no multiphase fluid in vessel 110), and first level indicator 106 may detect when separation vessel 110 is full (e.g., vessel full to capacity with the discrete sample of multiphase fluid, as illustrated in FIG. 1). Additional level indicators (not shown) may be installed in separation chamber 110 to detect intermediate fill levels (e.g., between full and empty) of vessel 110.

Control unit 180 may be configured to control operations of pump assembly 117 and/or control valve 118 based on sensor data indicating the fill level of separation vessel 110 received from first and second level indicators 106 and 107. For example, in response to receiving sensor data from first level indicator 106 indicating that inner chamber 112 is full with the discrete sample (e.g., measured amount) of multiphase fluid, control unit 180 may be configured to control operations of pump assembly 117 and/or control valve 118 to stop further flow of the multiphase fluid from holding chamber 115 into separation vessel 110. Similarly, in response to receiving sensor data from second level indicator 107 indicating that inner chamber 112 of separation vessel 110 is empty, control unit 180 may be configured to control operations of pump assembly 117 and/or control valve 118 to start flow of the multiphase fluid from holding chamber 115 into separation vessel 110 to fill inner chamber 112 with a discrete sample of the multiphase fluid that needs to be analyzed. First and second level indicators 106 and 107 can be devices suitable for indicating the level of liquid held in the inner chamber of separation vessel 110, such as sensors, a window, a float, and the like. Although FIG. 1 shows two level indicators 106 and 107, a person of ordinary skill will appreciate that some embodiments can use a single level indicator, and others may use more than two level indicators.

The multiphase fluid, delivered via multiphase fluid coupling 116 to inner chamber 112, can be generally characterized as a fluid that includes a mixture of at least an aqueous liquid phase (e.g., produced water) and a nonpolar liquid phase (e.g., crude oil). Analyzing the discrete sample contained in separation vessel 110 allows greater control over the separation of aqueous liquid and nonpolar liquid phases than could be achieved using a continuous process. In some embodiments, the multiphase fluid can include aqueous liquid droplets dispersed in the nonpolar liquid phase, nonpolar liquid droplets dispersed in the aqueous liquid phase, or both. The multiphase fluid can include an emulsion of aqueous liquid droplets emulsified in the nonpolar liquid phase, nonpolar liquid phase droplets emulsified in the aqueous liquid phase, or both. The aqueous liquid phase can include produced water from a corresponding well or group of wells. The nonpolar liquid phase can include crude oil produced from a corresponding well or group of wells. The multiphase fluid can contain between about 5 and 95 vol % nonpolar liquid phase and between about 5 and 95 vol % aqueous liquid phase. If the multiphase fluid contains less than about 5 vol % aqueous liquid phase there may not be a sufficient amount of water in the discrete sample received and contained in inner chamber 112 to separate it out and carry out in-situ analysis of geophysical properties thereof in built-in water analysis unit 140. According to at least one embodiment, the multiphase fluid can have a volume ratio of nonpolar liquid phase to aqueous liquid phase that is between about 99:1 and 30:70, alternately between about 95:5 and 40:60. In one or more embodiments, the multiphase fluid includes a gas phase. The gas phase can include gases produced from a corresponding well or group of wells, such as hydrocarbons, carbon oxides, hydrogen sulfide, mercaptans, and the like. The gas phase can be dissolved in the liquid phases of the multiphase fluid when it is introduced to separation vessel 110.

As explained previously, the multiphase fluid in separation vessel 110 can be a fluid obtained from a well or a group of wells. Alternately, the multiphase fluid in separation vessel 110 may be a multiphase fluid that has at least partially been treated upstream for separation of one or more of oil, water, and gas, after the extraction of the multiphase fluid from a well or a group of wells. For example, the multiphase fluid may be a multiphase fluid that has been processed at an upstream stage (upstream to separation vessel 110) to remove dissolved oil and/or gases. As inner chamber 112 is filled with the multiphase fluid, gases displaced by the multiphase fluid exit inner chamber 112 from gas outlet 113 to gas flow line 119. Gas flow line 119 can also be used to vent gases that come out of the multiphase fluid during or after filling separation vessel 110 and during the separation operation of the various liquid phases from the multiphase fluid filled in inner chamber 112. Gas flow meter 120 may be disposed on gas flow line 119 to measure the displaced or vented gas as it exits separation vessel 110. In some embodiments, control unit 180 may be communicatively coupled to flow meter 120 to obtain a measurement of gas exiting separation vessel.

As shown in FIG. 1, system 100 further includes demulsifier source 125 that may include one or more containers or vessels (e.g., reservoirs, tanks, tubes, injectors, and the like) suitable for storing one or more types of demulsifiers. Separation vessel 110 has demulsifier inlet 103, and demulsifier source 125 may be fluidly coupled to demulsifier inlet 103 via demulsifier coupling 126 to supply a measured (known or determined) amount and a determined type of demulsifier from demulsifier source 125 to separation vessel 110, based on the characteristics of the multiphase fluid contained in separation vessel 110, under control of control unit 180. For example, control unit 180 may be configured to determine, based on known characteristics of the discrete sample of multiphase fluid in separation vessel 110, the appropriate amount and type of demulsifier (from among a plurality of types of demulsifiers stored in source 125) to be used for introduction into separation vessel 110 and mixed with the multiphase fluid therein, so that an optimal or adequate level of separation between liquid phases including the aqueous liquid phase and the nonpolar liquid phase of the multiphase fluid in separation vessel 110 can be achieved.

Pump assembly 127A, demulsifier control valve 127B, and additional sensors (e.g., flow meters; not shown) may be disposed on demulsifier coupling 126 to introduce the measured amount and the predetermined type of demulsifier from demulsifier source 125 into separation vessel 110, under control of control unit 180. Pump assembly 127A may be driven by one or more electric motors. System 100 may further include one or more drives (e.g., VFDs; not shown) that monitor and control the electric motors under control of control unit 180. The control drives of pump assembly 127A, demulsifier control valve 127B, flow sensors (not shown), and control unit 180 may together define a control system for automatically introducing a measured amount and a predetermined type of demulsifier from source 125 into separation vessel 110 based on characteristics of the discrete sample of multiphase fluid contained therein.

The introduced measured amount and type of demulsifier from source 125 may be mixed with the multiphase fluid in inner chamber 112 to obtain a demulsified multiphase fluid. In some embodiments, control unit 180 may be configured to mix the selected amount and type of demulsifier with the multiphase fluid before the mixture is introduced into inner chamber 112.

Alternately, control unit 180 may actively mix the demulsifier with the multiphase fluid using mixer 108. FIG. 1 shows that mixer 108 is disposed inside separation vessel 110 at a bottom surface thereof. However, this is not intended to be limiting. Any type or number of mixers may be employed at any appropriate location inside or outside separation vessel 110 so long as the desired effect of adequately mixing the demulsifier with the multiphase fluid filled in inner chamber 112 can be achieved. Control unit 180 may be configured to turn on mixer 108 for a predetermined amount of time (e.g., 5 minutes) after the demulsifier is added to the multiphase fluid in inner chamber 112 to adequately mix the demulsifier into the multiphase fluid.

The demulsifier can be any component, such as a surface-active agent, that facilitates the aggregation of dispersed droplets of the aqueous liquid phase or the nonpolar liquid phase. Control unit 180 may be configured to automatically select the type (and amount) of demulsifier based on the type of crude oil and the amount of produced water that is typically produced from the multiphase fluid inside separation vessel 110 where the demulsifier is to be added. Nonlimiting examples of suitable demulsifiers include: polyol block copolymers, alkoxylated alkyl phenol formaldehyde resins, epoxy resin alkoxylates, amine-initiated polyol block copolymers, modified silicone polyethers, silicone polyethers, or similar components, and combinations of the same. Such demulsifiers are available from The Dow Chemical Company, Inc. and Ecolab, Inc. The amount and/or type of demulsifier that control unit 180 is configured to use can be an amount and/or type sufficient to facilitate the aggregation of dispersed droplets of the aqueous liquid phase or nonpolar liquid phase such that the bulk aqueous liquid phase and nonpolar liquid phase are separated. However, excess demulsifier can slow separation of the multiphase fluid and produce very stable emulsions. According to at least one embodiment, the amount of demulsifier control unit 180 is configured to use can be enough to produce a concentration of between about 1 and 100 ppmv demulsifier, alternately between about 1 and 50 ppmv, alternately between about 1 and 25 ppmv, alternately between about 5 and 10 ppmv.

After adding the demulsifier into the discrete sample of multiphase fluid in inner chamber 112 and mixing the demulsified multiphase fluid with mixer 108, control unit 180 is configured to allow the demulsified multiphase fluid to settle inside separation vessel 110 for a predetermined period of time, or until a predetermined condition of the demulsified multiphase fluid is achieved as determined based on data from one or more sensors (not shown). For example, the period of time can be predetermined to be between 1 minute and 24 hours, preferably between about 20 minutes and 12 hours, more preferably between about 1 and 5 hours. Also, the predetermined period of time may depend on the measured amount and type of demulsifier mixed into the multiphase fluid, and/or on the characteristics of the discrete sample of multiphase fluid in vessel 110. As a non-limiting example, the period of time can be predetermined to be approximately 2 hours. In this case, control unit 180 may be configured so that after adding the demulsifier into the multiphase fluid in separation vessel 110, control unit 180 may turn on mixer 108 for a predetermined amount of time (e.g., 5 minutes), and after passage of the predetermined amount of time, control unit 180 may control to turn off mixer 108, and allow the mixed demulsified multiphase fluid in separation vessel 110 to stabilize and settle for a predetermined period of time. For example, after turning off mixer 108, control unit 180 may start a timer and may determine that the demulsified multiphase fluid has adequately separated into constituent liquid phases including a separated nonpolar liquid phase and a separated aqueous liquid phase (i.e., separation operation complete) after the predetermined period of time has elapsed (e.g., after 2 hours).

In another embodiment, separation vessel 110 may be equipped with one or more sensors (not shown) that may be configured to detect sensor data, and control unit 180 may be configured to receive the sensor data and make a determination based on the sensor data as to whether the separation operation has completed. FIG. 1 illustrates a state of the demulsified multiphase fluid where the separation operation has already completed. That is, separation vessel 110 in FIG. 1 shows that the demulsified multiphase fluid has adequately separated into liquid phases including a separated nonpolar liquid phase 104 and a separated aqueous liquid phase 105 (e.g., countdown of the predetermined period of time has ended).

As shown in FIG. 1, system 100 further includes water analysis unit 140 that is disposed (e.g., built-in, installed, contained) inside separation vessel 110 and that includes analytical cell 141 defining inner space 142 (e.g., second inner chamber) where a sample of the produced water separated in inner chamber 112 can be channeled and contained. Built-in analytical cell 141 has sample inlet 114 in fluid communication with inner chamber 112 via sample coupling 132 to receive, as an aqueous liquid phase sample from inner chamber 112, a measured amount of the separated aqueous liquid phase after adequate separation thereof from the demulsified multiphase fluid (e.g., after completion of the separation operation). Sample control valve 129 may be disposed or installed on sample coupling 132 to selectively start, stop, and control a flow rate of a stream of the aqueous liquid phase sample flowing through sample coupling 132 from inner chamber 112 into inner space 142 of built-in analytical cell 141, based on control operations of control unit 180. Sample control valve 129 and control unit 180 may together define a control system for automatically and selectively controlling (e.g., starting, stopping, changing flow rate, and the like) a flow of a measured amount of the aqueous liquid phase (e.g., aqueous liquid phase sample) from inner chamber 112 to inner space 142.

System 100 further includes fresh water reservoir (e.g., fresh water source) 135 which stores fresh water (e.g., deionized water). Fresh water reservoir 135 includes an outlet that is in fluid communication with fresh water inlet 128 of built-in analytical cell 141 via fresh water coupling 130. As shown in FIG. 1, pump assembly 131 and fresh water control valve 134 may be disposed on fresh water coupling 130 to selectively start, stop, and control a flow rate of a stream of fresh water flowing through fresh water coupling 130, under control of control unit 180. Pump assembly 131 may be driven by one or more electric motors. System 100 may further include one or more drives (e.g., VFDs; not shown) that monitor and control the electric motors, under control of control unit 180. The control drives, fresh water control valve 134, and control unit 180 may together define a control system for automatically controlling a flow of a measured amount of fresh water from fresh water reservoir 135 to inner space 142. Built-in analytical cell 141 shown in FIG. 1 is thus in fluid communication with both inner chamber 112 via sample inlet 114, and fresh water reservoir 135 via fresh water inlet 128, and is configured to receive the aqueous liquid phase sample from inner chamber 112 and receive the measured amount of fresh water from fresh water reservoir 135, under control of control unit 180.

As shown in FIG. 1, an opening of sample control valve 129 (from where the aqueous liquid phase in inner chamber 112 enters sample coupling 132) may be located in a region of inner chamber 112 where the aqueous liquid phase is likely to accumulate after separation thereof from other liquid phases of the multiphase fluid. In many cases, the aqueous liquid phase will be denser than the nonpolar liquid phase and as a result, will settle beneath the nonpolar liquid phase inside separation vessel 110. Therefore, as shown in FIG. 1, analytical cell 141 (and at least the opening of sample control valve 129) may be located in a lower portion of separation vessel 110 where the separated aqueous liquid phase is likely to accumulate.

During operation, when control unit 180 determines (e.g., based on passage of the predetermined period of time, or based on sensor data) that the demulsified multiphase fluid in inner chamber 112 has adequately separated into liquid phases including the separate nonpolar liquid phase and the separate aqueous liquid phase (e.g., as shown in FIG. 1), and when control unit 180 further determines that built-in water analysis unit 140 is ready to accept a new produced water sample for in-situ analysis and measurement, control unit 180 may be configured to control sample control valve 129 to allow (e.g., channel, introduce) a predetermined measured amount of the aqueous liquid phase (e.g., aqueous liquid phase sample; nondiluted aqueous liquid phase sample) contained in inner chamber 112 into inner space 142 via sample coupling 132 and sample inlet 114, and control unit 180 may further be configured to control fresh water control valve 134 and pump assembly 131 to draw a predetermined measured amount of fresh water from reservoir 135 via fresh water coupling 130, and cause the predetermined measured amount of fresh water to flow into inner space 142 from fresh water inlet 128. Thus, the measured amount of aqueous liquid phase received via sample inlet 114 and the measured amount of fresh water received via fresh water inlet 128 are mixed into a diluted aqueous liquid phase sample 105A in inner space 142 for in-situ analysis and measurement.

Control unit 180 may be configured to control sample control valve 129, any pump associated with sample control valve 129, fresh water control valve 134, and pump assembly 131, such that the separated aqueous liquid phase sample from inner chamber 112 is conveyed to inner space 142 via sample coupling 132 separately from and/or concurrently with conveyance of the predetermined amount of fresh water from fresh water reservoir 135 to inner space 142 of analytical cell 141 via fresh water coupling 130. Further, although FIG. 1 shows inlets 114 and 128 as being separate from each other, this may not necessarily be the case. In an alternate embodiment, inlets 114 and 128 may be the same, and may include a junction (e.g., manifold (not shown)), and control unit 180 may be configured to control sample control valve 129, any pump associated with sample control valve 129, fresh water control valve 134, and pump assembly 131, such that a stream of the aqueous liquid phase sample received via sample coupling 132 mixes with a stream of the predetermined amount of fresh water received via fresh water coupling 130 at the junction, and thereby generate the diluted aqueous liquid phase sample prior to it being introduced into inner space 142 of analytical cell 141.

Thus, control unit 180 may control sample control valve 129 to deliver the predetermined measured amount (i.e., mass, volume, or both) of the aqueous liquid phase as the aqueous liquid phase sample that is to be mixed with the fresh water prior to the in-situ analysis and measurement. For example, control unit 180 may utilize data from one or more sensors (e.g., flow meters; not shown) disposed on sample coupling 132 to deliver the aqueous liquid phase sample having the measured amount to inner space 142 of analytical cell 141. Similarly, control unit 180 may control fresh water control valve 134 and pump assembly 131 to deliver the predetermined measured amount (i.e., mass, volume, or both) of the fresh water as the predetermined amount of fresh water to dilute the aqueous liquid phase sample and generate the diluted aqueous liquid phase sample 105A. For example, control unit 180 may utilize data from one or more sensors (e.g., flow meters; not shown) disposed on fresh water coupling 130 to deliver the fresh water having the measured amount to inner space 142 of analytical cell 141. Mixing of the measured amounts of aqueous liquid phase and fresh water to generate the diluted aqueous liquid phase sample may occur inside, outside, or partially inside and partially outside inner space 142 of analytical cell 141.

As shown in FIG. 1, water analysis unit 140 built-in inside separation vessel 110 includes one or more miniaturized probes (e.g., sensors, electrodes) 160 (e.g., 160A-160C) for measuring one or more physical or chemical properties of the diluted aqueous liquid phase sample. The properties measured may include total dissolved solids (TDS), salinity, pH, conductivity, sodium concentration, chloride concentration, sulfate concentration, carbonate concentration, nitrate concentration, and the like. Each probe 160 disposed in inner space 142 of analytical cell 141 may include an ion-selective electrode. Each probe 160 may have a stainless-steel body and have a sensing area (e.g., sensing region, sensing section, sensor tip) 170 (e.g., 170A-170C) at a tip of the probe that is adapted to be immersed in and come into contact with the diluted aqueous liquid phase sample contained in inner space 142 of analytical cell 141 to measure in-situ the one or more physical or chemical properties of the diluted aqueous liquid phase sample. A surface of each sensing area 170 of each probe 160 may be coated with an ion-exchange membrane. The membrane coating may provide ruggedness, protect the sensing area from corrosion, and prevent fouling of sensing area 170.

As shown in FIG. 1, sensor tips 170A-170C of probes 160A-160C may be positioned in inner space 142 of analytical cell 141 of water analysis unit 140 such that they can be immersed in the diluted aqueous liquid phase sample 105A after the sample has been introduced to inner space 142 from inner chamber 112. Probes 160A-160C may have an oblong shape with respective sensor tips 170A-170C located at a distal end. Probes 160A-160C can be oriented in a fixed position with respective sensor tips 170A-170C in a downward direction so that there exists an acute angle α measured from each probe 160 to a horizontal plane B. Orienting each probe 160 in this manner has the effect of allowing each probe 160 to be positioned so that corresponding sensor tip 170 can be positioned and immersed in the diluted aqueous liquid phase sample having a limited volume (e.g., volume that is insufficient to completely fill analytical cell 141 as shown in FIG. 1). Also, compared with probes oriented in a vertical or horizontal direction, orienting probes 160A-160C at an acute angle has the effect of reducing the accumulation of oil droplets near sensor tips 170A-170C, thereby preventing fouling and requiring less frequent maintenance and cleaning. The acute angle α can be between about 80° and 10°, preferably between about 60° and 30°. The acute angle α can vary based on the wall of analytical cell 141 or based on the shape of analytical cell 141 of water analysis unit 140. In at least one embodiment, the acute angle α is 45°.

The amount of fresh water used to dilute the aqueous liquid phase sample can be predetermined based on preset criteria (e.g., type of multiphase fluid from which the aqueous liquid phase has been separated, application requirements, sensing capacity of probes in water analysis unit 140, number of probes, fluid sample size contained in the analytical cell, and the like). For example, the ratio of fresh water to aqueous liquid phase in the diluted aqueous liquid phase sample can be between about 50:1 and 1:1, preferably between about 30:1 and 1:1, more preferably between about 10:1 and 15:1. As a specific (non-limiting) example, the ratio of fresh water to aqueous liquid phase in the diluted aqueous liquid phase sample that is contained in inner space 142 is 10:1.

Diluting the aqueous liquid phase sample with fresh water ensures that the capacity of each probe 160 for performing in-situ measurement is not overloaded, and increases the volume of the relatively small quantity of the aqueous liquid phase sample so that the sample can be analyzed by each probe 160 and adequately immerse each probe 160 disposed in series inside inner space 142. That is, the dilution step (e.g., diluting the produced water sample with a sample of fresh water by 10 times) enables application of multiple ion-selective electrodes for in-situ measurement of properties of the produced water sample in series inside inner space 142, while also ensuring that the measured properties remain within the specified operating range of ion-selective electrodes 160. This step can also reduce the corrosive potential of the aqueous liquid phase sample, allowing system 100 components to be manufactured from materials which might otherwise be unsuitable.

As explained previously, inner space 142 defined by built-in analytical cell 141 is in fluid communication with inner chamber 112 and fresh water reservoir 135 via sample inlet 132 and fresh water inlet 128, respectively. In FIG. 1, inner space 142 is defined in a bottom region of inner chamber 112. That is, analytical cell 141 of water analysis unit 140 is disposed at a bottom of separation vessel 110. This is not intended to be limiting. Inner space 142 may be defined (and analytical cell 141 may be disposed) and/or the opening of sample control valve 129 may be positioned, at any suitable location or region of inner chamber 112 and separation vessel 110, so long as the aqueous liquid phase separated from the multiphase fluid and contained inside inner chamber 112 can be flown into analytical cell 141 via sample inlet 114 efficiently.

Further, in FIG. 1, analytical cell 141 of water analysis unit 140 has a rectangular shape. However, in other embodiments, analytical cell 141 may have a shape that narrows toward a minimum point (e.g., a funnel shape, conical shape, rounded bottom, and the like). Shape of inner space 142 of analytical cell 141 is not intended to be limiting, so long as the shape provides a suitable depth of the diluted aqueous liquid phase sample contained therein, so that each sensor tip 170 of each probe 160 disposed in inner space 142 can be adequately immersed in and come into contact with the diluted aqueous liquid phase sample for in-situ analysis and measurement, without requiring large volumes of the diluted aqueous liquid phase sample.

Further, the number of probes 160 inside inner space 142 of built-in water analysis unit 140 is not intended to be limiting. As a non-limiting example, FIG. 1 illustrates water analysis unit 140 built-in inside separation vessel 110 including probes 160A, 160B, and 160C located proximally (e.g., adjacent or next to each other) inside inner space 142. In other embodiments, one, or two or four or more probes can be disposed inside inner space 142. The number of probes 160 and the type each probe included in built-in water analysis unit 140 may be determined based on the particular application requirements. Further, the number of analytical cells 141 of water analysis units 140 disposed inside separation vessel 110 may be more than one and may also be determined based on particular application requirements. In some embodiments, each probe 160 may be disposed in a separate analytical cell 141. For example, a first probe 160 may be disposed in a first cell 141 disposed inside inner chamber 112, a second probe 160 may be disposed in a second cell 141 disposed inside inner chamber 112, and so on. Thus, separation vessel 110 may include one or more built-in analytical cells 141, each with a corresponding number of (one or more) probes 160. Any suitable configuration of analytical cell(s) 141 and probe(s) inside the analytical cells can be deployed inside separation vessel 110 so long as desired geophysical or geochemical properties of the diluted aqueous liquid phase sample corresponding to separation vessel 110 can be measured in-situ and recorded. Further, in the embodiment shown in FIG. 1, system 100 includes a single separation vessel 110. This is not intended to be limiting. Other embodiments of system 100 may include multiple separation vessels 110, each including corresponding components as described above and in connection with FIG. 1. In summary, the size, shape, and number of separation vessel 110, built-in analytical cell 141, probes 160, and sensor tips 170, are not intended to be limiting to what is illustrated in FIG. 1. Any suitable size, shape, and number separation vessel 110, built-in analytical cell 141, probes 160, and sensor tips 170, may be employed so long as in-situ analysis and measurement of desired one or more physical or chemical properties of each diluted aqueous liquid phase sample can be obtained inside the chamber where the aqueous liquid phase sample was separated.

As explained previously, sensor tip 170 of each electrode or probe 160 may be coated with an ion-exchange membrane to prevent accumulation of oil at or near the sensing area. Even when present in extremely limited quantities, oil in the diluted aqueous liquid phase sample can foul sensing area 170 of probes 160 and cause inaccurate measurements. The membrane coating helps prevent the accumulation of oil droplets at or near sensing area 170. The ion-exchange membrane used for coating sensing area 170 may be a polar material directly applied to the surface of sensing area 170 of each electrode 160 to allow exchange of the produced water sample but prevent oil droplets from sticking to the sensing area surface of each electrode 160. The polar material of the ion-exchange membrane may be any material that is sufficiently permeable and suitable for coating sensing area 170 that is to be used in an aqueous environment, so as to allow the diluted aqueous liquid phase sample to contact the surface of sensing area 170 of each probe 160, while blocking any residual oil from contacting sensing area 170. For example, the polar material can include a polymer such as polyvinyl acetate, polyimide, polybenzimidazole, polyacrylonitrile, polyethersulfone, sulfonated tetrafluoroethylene-based fluoropolymer-copolymer, or similar materials, and combinations of the same.

During operation, control unit 180 controls components of system 100 consistent with the manner described above to introduce (channel, flow, convey) the diluted aqueous liquid phase sample in inner space 142 of built-in analytical cell 141 of water analysis unit 140 to fill analytical cell 141 (e.g., as shown in FIG. 1) with the diluted aqueous liquid phase sample such that sensor tips 170 (e.g., each of 170A, 170B, and 170C) of probes 160 (e.g., each of 160A, 160B, and 160C) are immersed in and come in predetermined contact with the diluted aqueous liquid phase sample filled in inner space 142. Control unit 180 may further be configured to control components of system 100 so that the diluted aqueous liquid phase sample in inner space 142 remains in contact with respective sensor tips 170 of probes 160 during an in-situ measurement operation for a predetermined period of time. The predetermined period of time may be preset (e.g., approximately 10 or 15 minutes), or may be determined based on predetermined logic of control unit 180.

For example, the preset period of time of the in-situ measurement operation can be predetermined to be between 30 seconds and 1 hour, preferably between about 1 minute and about 20 minutes, more preferably between about 3 minutes and 5 minutes. At the end of the preset period, control unit 180 may control to obtain measurement data from the one or more probes 160 and record the data in memory. Alternately, control unit 180 may be configured to detect when the measurement or output of the one or more probes 160 has adequately stabilized so as to determine that a steady reading from sensors 160 has been obtained. In this case, control unit 180 may be configured to maintain the diluted aqueous liquid phase sample in inner space 142 in contact with respective sensor tips 170 of the one or more probes 160 until the steady reading has been detected and recorded. Once control unit 180 detects the stable reading or once the preset period of time has elapsed, control unit 180 stores in memory, a set of measurement data corresponding to the output of the one or more probes 160 as diluted aqueous liquid phase sample data. Control unit 180 may record the diluted aqueous liquid phase sample data in association with other relevant data such as data regarding the multiphase fluid in inner chamber 112 from which the aqueous liquid phase sample for in-situ measurement was drawn, demulsifier data, source well information, and the like.

Control unit 180 may further be configured to calculate approximate corresponding values from the diluted aqueous liquid phase sample data for the nondiluted aqueous liquid phase sample extracted from inner chamber 112 by adjusting the diluted aqueous liquid phase sample data to account for the measured amount of dilution with fresh water from fresh water reservoir 135. That is, control unit 180 may be configured to calculate and record in memory, a set of measurement data corresponding to the nondiluted aqueous liquid phase sample as the nondiluted aqueous liquid phase sample data (i.e., aqueous liquid phase sample data), based on the recorded set of measurement data corresponding to diluted aqueous liquid phase sample, and based on data regarding the ratio of fresh water to aqueous liquid phase in the diluted aqueous liquid phase sample. Control unit 180 can also be configured to adjust the calculated nondiluted aqueous liquid phase sample data to account for properties of the fresh water used for the dilution. For example, if the property to be approximated is the concentration of a solute, the processing unit 180 can be configured to adjust the calculated nondiluted aqueous liquid phase sample data to account for a known preexisting concentration of the solute in the fresh water that is used to dilute the aqueous liquid phase sample.

As shown in FIG. 1, system 100 may further include MPFM 190 that is communicatively coupled to control unit 180. MPFM 190 may be used to measure the flow rate of each phase of a multiphase fluid (e.g., including at least oil and produced water) of a well or a group of wells at a GOSP or at a well site. Control unit 180 may be configured to transmit the calculated values corresponding to the nondiluted aqueous liquid phase sample data (e.g., (adjusted or recorded) set of measurement data corresponding to nondiluted aqueous liquid phase sample) to MPFM 190 to calibrate, optimize, or control MPFM 190, so that MPFM 190 can detect flow rate of oil in the multiphase fluid passing therethrough more accurately.

Further, as shown in FIG. 1, analytical cell 141 disposed (built-in) inside inner chamber 112 further has sample outlet 193 in fluid communication with drain equipment 195 via water analysis coupling 191. Water analysis control valve 192 may be disposed on water analysis coupling 191 to selectively start, stop, and control a flow rate of a stream of the diluted aqueous liquid phase sample (or fresh water) being drained out of inner space 142 of analytical cell 141, under control of control unit 180. After the diluted aqueous liquid phase sample contained in inner space 142 has been analyzed by the one or more probes 160 and corresponding diluted aqueous liquid phase sample data recorded, control unit 180 may control water analysis control valve 192 to remove (drain) the diluted aqueous liquid phase sample from inner space 142 via water analysis coupling 191 to drain equipment 195. Although not shown in FIG. 1, system 100 may include a pump assembly to remove the diluted aqueous liquid phase sample from analytical cell 141 of water analysis unit 140 and flow the diluted aqueous liquid phase sample to drain equipment 195. Water analysis control valve 192, control drives (if any) and control unit 180 may together define a control system for automatically controlling draining of the fluid out of inner space 142. After draining the diluted aqueous liquid phase sample, control unit 180 may further be configured to control pump assembly 131 and control valve 134 to flow fresh water from fresh water reservoir 135 into inner space 142 to thereby flush (e.g., rinse) inner space 142 with fresh water, clean sensor tips 170, and prepare water analysis unit 140 to receive subsequent samples without any cross contamination between the samples.

Further, as shown in FIG. 1, separation vessel 110 has drain outlet 194 to fluidly communicate inner chamber 112 with drain equipment 195 via drain coupling 197 to drain the demulsified multiphase fluid in inner chamber 112, after the aqueous liquid phase sample has been extracted therefrom via sample coupling 132 of water analysis unit 140, or after the corresponding diluted (or nondiluted) aqueous liquid phase sample data has been recorded by control unit 180 in memory (FIG. 3). Drain control valve 196 may be disposed on drain coupling 197 to selectively start, stop, and control a flow rate of a stream of the fluid being drained out of inner chamber 112, under control of control unit 180. Drain control valve 196, pump control drive (if any), and control unit 180 may together define a control system for automatically controlling draining of the fluid out of inner chamber 112. Thus, after the diluted aqueous liquid phase sample has been analyzed by the one or more sensors 160 and corresponding diluted aqueous liquid phase sample data recorded (or after the corresponding aqueous liquid phase sample has been drawn from inner chamber 112 and channeled into inner space 142 for in-situ analysis), control unit 180 may control drain control valve 196 to drain the fluid from inner chamber 112 via drain coupling 197 to drain equipment 195, and prepare emptied separation vessel 110 for a next sample of multiphase fluid. After emptying (e.g., based on second level indicator 107 indicating that inner chamber 112 is empty) separation vessel 110, control unit 180 may also be configured to flush (e.g., rinse) inner chamber 112 with fresh water from reservoir 135 in preparation for receiving a next discrete sample of multiphase fluid.

The above process of system 100 thus repeats with each new discrete sample of the multiphase fluid introduced into separation vessel 110 after in-situ analysis and measurement for a previous discrete sample of the multiphase fluid has been completed. The process can be automated by control unit 180 so that discrete samples of multiphase fluid in separation vessel 110 are continuously subject to in-situ analysis and measurement in real-time, sets of measurement data recorded in memory, and the data transmitted to MPFM 190 for calibrating, optimizing, or controlling accuracy of data output from MPFM 190 with minimal or no supervision. The automation allows direct feeding of data to the MPFM to streamline and expedite the process of well monitoring, while reducing error. The system 100 can thus be used to analyze discrete multiphase fluid samples from one or more wells, allowing less-productive wells to be identified and isolated.

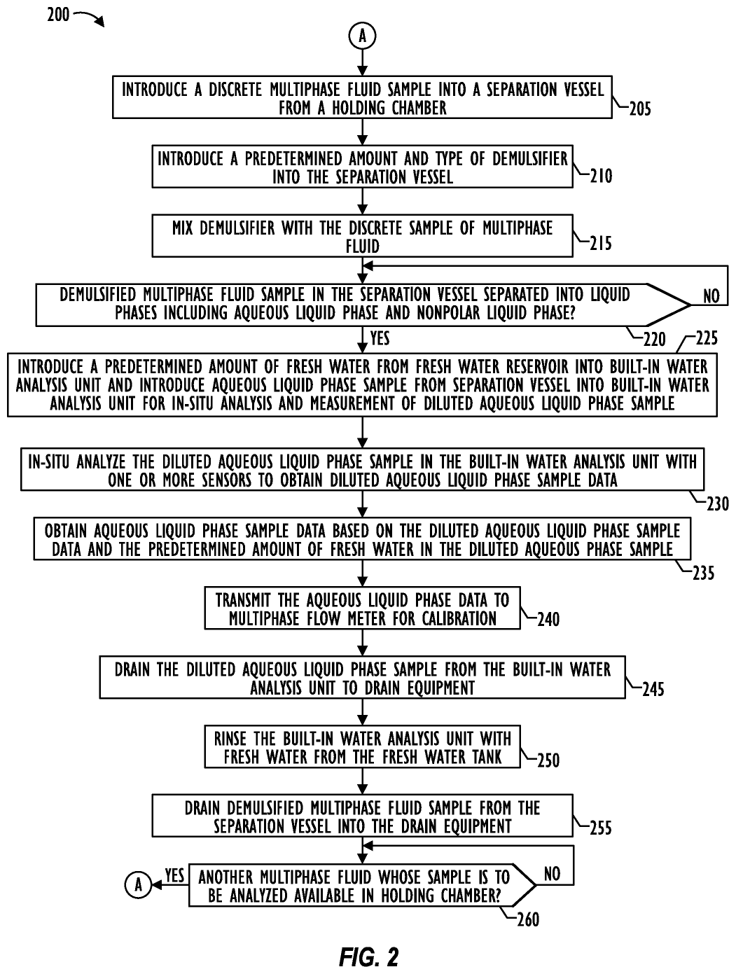

FIG. 2 is a flow chart that illustrates method 200 of operation of the system illustrated in FIG. 1, in accordance with one or more embodiments. Method 200 begins at block 205 where a discrete sample of multiphase fluid is introduced into separation vessel 110 from holding chamber 115. At block 205, in response to control unit 180 determining (e.g., based on data received from corresponding first and second level indicators 106 and 107) that separation vessel 110 is in an empty state, and also determining (e.g., based on sensor data) that holding chamber 115 contains multiphase fluid that needs to be analyzed and its sensor data measured in-situ, control unit 180 may control pump assembly 117 and inlet control valve 118 disposed on multiphase fluid coupling 116 of separation vessel 110 to permit a discrete sample of the multiphase fluid in holding chamber 115 to flow into and fill inner chamber 112 of separation vessel 110. For example, control unit 180 may control to continue the filling operation until level indicators 106 and 107 indicate that separation vessel 110 is in a full state. The discrete sample of multiphase fluid may be associated with a selected well or a selected group of wells whose produced water sample needs to be analyzed to measure properties thereof in-situ, and calibrate, control, or operate the MPFM based on the in-situ measurement.

Method 200 then proceeds to block 210 where control unit 180 controls pump assembly 127A and control valve 127B to introduce a predetermined measured amount and type of demulsifier from demulsifier source 125 into separation vessel 110. At block 210, control unit 180 is configured to determine the measured amount and type of demulsifier to be introduced into inner chamber 112 based on predetermined data representing the type of crude oil and the amount of produced water that is typically produced from the multiphase fluid inside separation vessel 110 filled at block 205. At block 215, control unit 180 controls mixer 108 to mix the demulsifier with the multiphase fluid inside inner chamber 112 (i.e., mixing operation). At block 215, control unit 180 may operate mixer 108 for a predetermined period of time (e.g., 5 minutes) after the demulsifier is added to the multiphase fluid in separation vessel 110 at block 210.

Method 200 then proceeds to block 220 where control unit 180 determines whether the discrete sample of multiphase fluid contained in inner chamber 112 has adequately separated into liquid phases including a separate aqueous liquid phase and a separate nonporous liquid phase. At block 220, control unit 180 may be configured to determine that adequate separation has been achieved (e.g., separation operation completed) based on passage of the predetermined period of time since completion of the mixing operation at block 215. For example, control unit 180 may determine that the separation operation has completed when approximately 2 hours have elapsed since completion of the mixing operation. Alternately, or in addition, control unit 180 at block 220 may be configured to determine that the separation operation has completed based on sensor data from one or more sensors (not shown; e.g., optical sensors, conductivity sensors, and the like) disposed in separation vessel 110 making such a determination.

In response to control unit 180 determining that the discrete sample in inner chamber 112 has adequately separated into liquid phases including the separate aqueous liquid phase and the separate nonporous liquid phase (YES at block 220; separation operation complete), method 200 proceeds to block 225 where control unit 180 controls pump assembly 131 and fresh water control valve 134 to introduce (e.g., channel, flow, convey) a measured amount of fresh water from fresh water reservoir 135 into inner space 142 inside inner chamber 112 of separation vessel 110, and further control sample control valve 129, and any associated pump, to introduce (e.g., channel, flow, convey) a measured amount (e.g., 50-60 milliliters) of the separated aqueous liquid phase (e.g., aqueous liquid phase sample) from inner chamber 112 into inner space 142, for in-situ analysis and measurement of the diluted aqueous liquid phase sample.

At block 225, control unit 180 may be configured draw the measured amount of the aqueous liquid phase as the nondiluted aqueous liquid phase sample from inner chamber 112, and draw the measured amount of fresh water from fresh water reservoir 135, using one or more sensors (e.g., flow meters), so that the nondiluted aqueous liquid phase sample and the fresh water are mixed at a predetermined ratio (e.g., 10:1) to generate the diluted aqueous liquid phase sample. As explained previously, the mixing and resultant generation of the diluted aqueous liquid phase sample may occur outside, inside, or partially inside and partially outside inner space 142 of analytical cell 141. For example, at block 225, control unit 180 may be configured so that first, the measured amount of fresh water is channeled into inner space 142, and second, the nondiluted aqueous liquid phase sample is channeled into inner space 142, so that the mixing and resultant generation of the diluted aqueous liquid phase sample occurs inside inner space 142 of analytical cell 141.

Operations of block 225 are further illustrated by way of example with reference to FIG. 1. Since the discrete sample of multiphase fluid in separation vessel 110 has adequately separated into liquid phases including separate aqueous liquid phase 105 and separate nonporous liquid phase 104 (e.g., control unit determined that the corresponding predetermined period of time (e.g., 2 hours) has elapsed since end of the active mixing operation), control unit 180 at block 225 operates pump assembly 131 and fresh water control valve 134 to draw a measured amount of fresh water from reservoir 135, and at the same time, control unit 180 operates sample control valve 129 to allow a measured amount of separated aqueous liquid phase 105 which has accumulated at the bottom of inner chamber 112 of separation vessel 110 to flow into inner space 142 of water analysis unit 140 as the separated aqueous liquid phase sample. As a result, an aqueous liquid phase sample stream flowing into inner space 142 via sample inlet 114 combines and mixes with a fresh water stream flowing into inner space 142 via fresh water inlet 128 to generate the diluted aqueous liquid phase sample 105A in inner space 142. At block 225, control unit 180 controls to fill inner space 142 of analytical cell 141 of water analysis unit 140 with the diluted aqueous liquid phase sample 105A having a measured total amount so that the diluted aqueous liquid phase sample 105A comes in predetermined contact with sensor tips 170 of one or more probes 160 disposed inside inner space 142. That is, control unit 180 controls to fill analytical cell 141 with the diluted aqueous liquid phase sample so that, as shown in FIG. 1, sensor tips 170 are completely immersed in and maintain predetermined contact with the diluted aqueous liquid phase sample during the in-situ analysis and measurement of the diluted aqueous liquid phase sample.

Method 200 then proceeds to block 230 where the diluted aqueous liquid phase sample 105A contained in inner space 142 of analytical cell 141 of water analysis unit 140 is in-situ analyzed with the at least one probe 160 to obtain diluted aqueous liquid phase sample data (e.g., set of measurement data corresponding to diluted aqueous liquid phase sample). At block 235, control unit 180 accounts for the dilution of the aqueous liquid phase sample by performing predetermined processing on the diluted aqueous liquid phase sample data to obtain nondiluted aqueous liquid phase sample data (e.g., set of measurement data corresponding to nondiluted aqueous liquid phase sample).

Continuing with the above example of FIG. 1, control unit 180 at block 230 may transmit control signals to the one or more probes 160 to cause the probes 160 to continuously measure and transmit sensor data to control unit 180. Control unit 180 may be configured to maintain the predetermined contact in inner space 142 between probes 160 and the diluted aqueous liquid phase sample until passage of the preset period of time, or until the continuously received sensor data from the one or more sensors 160 stabilizes, thereby indicating that a steady reading has been obtained. The control unit 180 may be configured to record in memory the stabilized sensor data as diluted aqueous liquid phase sample data (e.g., set of measurement data corresponding to diluted aqueous liquid phase sample). Further, control unit 180 is configured to perform predetermined operations (e.g., account for the fresh water added to the aqueous liquid phase sample) on the diluted aqueous liquid phase sample data to obtain sensor data corresponding to the nondiluted aqueous liquid phase sample obtained from inner chamber 112 of separation vessel 110 (e.g., set of measurement data corresponding to nondiluted aqueous liquid phase sample). For example, control unit 180 may control to allow 1:10 dilution (v/v) to take place, i.e. ten times dilution of the produced water sample by adding fresh water. Once the diluted aqueous liquid phase sample data corresponding to the 1:10 diluted produced water sample is obtained, control unit 180 multiplies the measured values by ten times to correct for the dilution effect, and obtain back calculated values as the nondiluted aqueous liquid phase sample data corresponding to the nondiluted aqueous liquid phase sample. Control unit 180 may store the nondiluted aqueous liquid phase sample data in memory, along with corresponding data regarding the multiphase fluid in separation vessel 110, and other corresponding data.

At block 240, control unit 180 may transmit the (nondiluted) aqueous liquid phase sample data obtained at block 235 to MPFM 190 to calibrate, optimize, or control MPFM 190 so that MPFM 190 can detect flow rates of oil in the multiphase fluid passing therethrough more accurately. As a result of method 200, MPFM 190 is able to more accurately detect the constituent flow rates of various liquid phases (e.g., crude oil, produced water) of the multiphase fluid that was analyzed in-situ at block 230 and that is flowing through MPFM 190. Method 200 then proceeds to block 245 where control unit 180 operates control valve 192 and/or pump assembly (not shown in FIG. 1) to drain the fluid from inner space 142 and into drain equipment 195. At block 250, control unit 180 may control pump assembly 131 and control valve 134 to flow fresh water from fresh water reservoir 135 into inner space 142 to rinse and prepare inner space 142 for a next diluted sample. For example, after draining the sample at block 245, control unit 180 may cause fresh water to fill inner space 142 and further drain the fresh water therefrom into drain equipment 195. This process may be performed one or more times, to prevent cross contamination between samples to be analyzed consecutively in-situ by water analysis unit 140, and also to clean sensor tips 170 to ensure accurate in-situ measurement of each water sample.

Next, at block 255, control unit 180 controls drain control valve 196 to drain the fluid contained inside inner chamber 112 to drain equipment 195. Continuing with the above example of FIG. 1, control unit 180 at block 255 controls drain control valve 196 to be in an open position (and optionally, drive a pump assembly (not shown)) to drain the fluid out of inner chamber 112 of separation vessel 110 via drain coupling 197 to drain equipment 195. At block 255, control unit 180 may continue to drain fluid out of inner chamber 112 until sensor data received from second level indicator 107 indicates that inner chamber 112 is empty. Control unit 180 at block 255 may also perform operations to rinse empty separation vessel 110 with fresh water from reservoir 135 prior to selectively filling inner chamber 112 of separation vessel 110 with a next discrete sample of multiphase fluid to prevent cross contamination between consecutive samples to be contained in inner chamber 112.

Method 200 next proceeds to block 260 where control unit 180 determines (e.g., based on sensor data associated with holding chamber 115) whether multiphase fluid whose sample needs to be analyzed by built-in water analysis unit 140 is present in holding chamber 115. In response to determining that a multiphase fluid whose sample needs to be analyzed is present in holding chamber 115 (YES at block 260), method 200 proceeds to block 205, and the steps of method 200 are repeated to analyze the new discrete sample of multiphase fluid. On the other hand, in response to determining that a multiphase fluid whose sample needs to be analyzed is not present in holding chamber 115 (NO at block 260), method 200 waits until a new sample becomes available in holding chamber 115 for analysis. At block 220, in response to control unit 180 determining that the discrete sample in inner chamber 112 has not adequately separated into liquid phases including the separate aqueous liquid phase and the separate nonporous liquid phase (NO at block 220; separation operation not complete), method 200 waits until the separation operation has completed.

In this manner, multiple samples are continuously and automatically analyzed by the system and method, and corresponding measurement data recorded automatically. Further, by providing built-in water analysis unit 140 inside inner chamber 112 of separation vessel 110 for in-situ measurement of the aqueous liquid phase sample data, a separate external analytical chamber is not required, and the automated analysis and measurement of the data can be performed in-situ, without having to flow the separated aqueous liquid phase sample from the bottom of separation vessel 110 to an external analysis unit, thereby simplifying operation, increasing efficiency, and reducing cost. The in-situ measurement technique disclosed herein may further prevent contamination of the separated aqueous liquid phase sample that may otherwise happen in case the aqueous liquid phase sample is to be flown to external analytical chamber for analysis, thereby ensuring or increasing accuracy of the measured aqueous liquid phase sample data.

FIG. 3 is a functional block diagram of an exemplary computer system (or “system”) 300 in accordance with one or more embodiments. In some embodiments, system 300 is a PLC, system on a chip, ASIC, and the like. System 300 may include memory 304, processor 306 and input/output (I/O) interface 308. Memory 304 may include non-volatile memory (e.g., flash memory, solid state memory, read-only memory (ROM), programmable read-only memory (PROM), erasable programmable read-only memory (EPROM), electrically erasable programmable read-only memory (EEPROM)), volatile memory (e.g., random access memory (RAM), static random access memory (SRAM), synchronous dynamic RAM (SDRAM)), or bulk storage memory (e.g., CD-ROM or DVD-ROM, hard drives). Memory 304 may include a non-transitory computer-readable storage medium (e.g., non-transitory program storage device) having program instructions 310 stored thereon. Program instructions 310 may include program modules 312 that are executable by a computer processor (e.g., processor 306) to cause the functional operations described herein, such as those described with regard to control unit 180, MPFM 190, or method 200.

Processor 306 may be any suitable processor capable of executing program instructions. Processor 306 may include a central processing unit (CPU) that carries out program instructions (e.g., the program instructions of the program modules 312) to perform the arithmetical, logical, or input/output operations described. Processor 306 may include one or more processors. I/O interface 308 may provide an interface for communication with one or more I/O devices 314, such as a joystick, a computer mouse, a keyboard, or a display screen (for example, an electronic display for displaying a graphical user interface (GUI)). I/O devices 314 may include one or more of the user input devices. I/O devices 314 may be connected to I/O interface 308 by way of a wired connection (e.g., an Industrial Ethernet connection) or a wireless connection (e.g., a Wi-Fi connection). I/O interface 308 may provide an interface for communication with one or more external devices 316. In some embodiments, I/O interface 308 includes one or both of an antenna and a transceiver. In some embodiments, external devices 316 include any of the electronic components communicatively coupled to control unit 180 and that are described above in connection with FIGS. 1 and 2.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the embodiments. It is to be understood that the forms of the embodiments shown and described herein are to be taken as examples of embodiments. Elements and materials may be substituted for those illustrated and described herein, parts and processes may be reversed or omitted, and certain features of the embodiments may be utilized independently, all as would be apparent to one skilled in the art after having the benefit of this description of the embodiments. Changes may be made in the elements described herein without departing from the spirit and scope of the embodiments as described in the following claims. Headings used herein are for organizational purposes only and are not meant to be used to limit the scope of the description.

It will be appreciated that the processes and methods described herein are example embodiments of processes and methods that may be employed in accordance with the techniques described herein. The processes and methods may be modified to facilitate variations of their implementation and use. The order of the processes and methods and the operations provided may be changed, and various elements may be added, reordered, combined, omitted, modified, and so forth. Portions of the processes and methods may be implemented in software, hardware, or a combination of software and hardware. Some or all of the portions of the processes and methods may be implemented by one or more of the processors/modules/applications described here.

As used throughout this application, the word “may” is used in a permissive sense (e.g., meaning having the potential to), rather than the mandatory sense (e.g., meaning must). The words “include,” “including,” and “includes” mean including, but not limited to. As used throughout this application, the singular forms “a”, “an,” and “the” include plural referents unless the content clearly indicates otherwise. Thus, for example, reference to “an element” may include a combination of two or more elements. As used throughout this application, the term “or” is used in an inclusive sense, unless indicated otherwise. That is, a description of an element including A or B may refer to the element including one or both of A and B. As used throughout this application, the phrase “based on” does not limit the associated operation to being solely based on a particular item. Thus, for example, processing “based on” data A may include processing based at least in part on data A and based at least in part on data B, unless the content clearly indicates otherwise. As used throughout this application, the term “from” does not limit the associated operation to being directly from. Thus, for example, receiving an item “from” an entity may include receiving an item directly from the entity or indirectly from the entity (e.g., by way of an intermediary entity). Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. In the context of this specification, a special purpose computer or a similar special purpose electronic processing/computing device is capable of manipulating or transforming signals, typically represented as physical, electronic or magnetic quantities within memories, registers, or other information storage devices, transmission devices, or display devices of the special purpose computer or similar special purpose electronic processing/computing device.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations may be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). The use of the term “about” means ±10% of the subsequent number, unless otherwise stated.

Use of the term “optionally” with respect to any element of a claim means that the element is required, or alternatively, the element is not required, both alternatives being within the scope of the claim. Use of broader terms such as comprises, includes, and having may be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise.

Many other embodiments will be apparent to those of skill in the art upon reviewing the above description. The scope of the subject matter of the present disclosure therefore should be determined with reference to the appended claims, along with the full scope of equivalents to which such claims are entitled. In the appended claims, the terms “including” and “in which” are used as the plain-English equivalents of the respective terms “comprising” and “wherein.”

What is claimed is:

1. A system for separating and in-situ analyzing a discrete sample of multiphase fluid, the system comprising:
a separation vessel having a first inner chamber for separating a discrete sample of multiphase fluid into liquid phases including an aqueous liquid phase and a nonporous liquid phase; and
a built-in water analysis unit including:
an analytical cell disposed inside the first inner chamber of the separation vessel, the analytical cell having a second inner chamber; and
at least one probe having a sensing area disposed in the second inner chamber for in-situ analysis of a sample of the aqueous liquid phase that is separated from the discrete sample of multiphase fluid in the first inner chamber and that is channeled to the second inner chamber from the first inner chamber for the in-situ analysis,
wherein the second inner chamber is defined inside the first inner chamber.

2. The system according to claim 1, wherein the at least one probe has an oblong shape, and wherein the sensing area of the probe is covered with an ion-exchange membrane to prevent fouling of the sensing area.

3. The system according to claim 1, wherein the analytical cell is built-in in a bottom portion of the separation vessel such that an opening of the sample control valve is disposed in a bottom region of the first inner chamber, where the aqueous liquid phase is likely to accumulate after separating from the discrete sample of multiphase fluid.

4. The system according to claim 1, wherein the analytical cell has a sample inlet and wherein the second inner chamber is in fluid communication with the first inner chamber via the sample inlet.

5. The system according to claim 4, wherein the built-in water analysis unit further includes a sample control valve coupled to the sample inlet for controlling a flow of the separate aqueous liquid phase from the first inner chamber to the second inner chamber,
wherein the analytical cell further has a fresh water inlet, and the second inner chamber is in fluid communication with a fresh water reservoir via the fresh water inlet, and
wherein the system further comprises one or more processors operatively coupled to the sample control valve and the at least one probe, the one or more processors being configured to:
control the sample control valve to channel a predetermined amount of the separate aqueous liquid phase as the aqueous liquid phase sample from the first inner chamber to the second inner chamber via the sample inlet;
dilute the aqueous liquid phase sample channeled into the second inner chamber with a predetermined amount of fresh water introduced into the second inner chamber via the fresh water inlet, to generate a diluted aqueous liquid phase sample;
in-situ analyze the diluted aqueous liquid phase sample in the second inner chamber with the at least one probe to obtain diluted aqueous liquid phase sample data;
calculate nondiluted aqueous liquid phase sample data based on the diluted aqueous liquid phase sample data, as well as based on the predetermined amount of fresh water in the diluted aqueous liquid phase sample; and
transmit the nondiluted aqueous liquid phase sample data to a multiphase flow meter for calibration.

6. The system according to claim 5, wherein the sensing area of the at least one probe is at a distal end of the probe, and wherein the probe is oriented in the second inner chamber such that the sensing area is immersed in the diluted aqueous liquid phase sample when the diluted aqueous liquid phase sample is contained in the second inner chamber.

7. The system according to claim 5, wherein the at least one probe includes an ion-selective electrode configured to in-situ measure one or more properties of the diluted aqueous liquid phase sample, the one or more properties selected from a group including: sodium concentration, chloride concentration, total dissolved solids (TDS) concentration, pH, conductivity, sulfate concentration, carbonate concentration, and nitrate concentration.

8. The water analysis unit according to claim 5, wherein the at least one probe includes first, second, and third probes that are proximally disposed adjacent to each other such that each probe is oriented in the second inner chamber with the sensing area of the probe in a downward direction and immersed in the diluted aqueous liquid phase sample when the diluted aqueous liquid phase sample is contained in the second inner chamber, and such that there exists an acute angle measured from the probe to a horizontal plane that is substantially perpendicular to a direction of gravity.

9. The water analysis unit according to claim 8, wherein the acute angle is in the range of 30°-60°.

10. The system according to claim 5, wherein the one or more processors are further configured to:
introduce the discrete sample of multiphase fluid into the first inner chamber of the separation vessel via a multiphase fluid inlet of the separation vessel;
mix a predetermined amount of demulsifier obtained from a demulsifier source with the discrete sample of multiphase fluid in the first inner chamber to cause the discrete sample to separate into liquid phases including the aqueous liquid phase and the nonpolar liquid phase; and
control the sample control valve to channel the predetermined amount of the aqueous liquid phase as the aqueous liquid phase sample from the first inner chamber to the second inner chamber via the sample inlet of the analytical cell, in response to determining that the discrete sample of multiphase fluid in the first inner chamber has separated into liquid phases including the aqueous liquid phase and the nonpolar liquid phase.

11. The system according to claim 5, wherein the analytical cell further has a sample outlet, wherein the separation vessel has a drain outlet, and wherein the one or more processors are further configured to:
drain the diluted aqueous liquid phase sample in the second inner chamber via the sample outlet after obtaining the diluted aqueous liquid phase sample data;
rinse the second inner chamber and the sensing area of the at least one probe disposed in the second inner chamber with fresh water introduced into the second inner chamber via the fresh water inlet after draining the diluted aqueous liquid phase sample; and
drain the discrete sample of multiphase fluid in the first inner chamber via the drain outlet after channeling the predetermined amount of the aqueous liquid phase as the aqueous liquid phase sample from the first inner chamber to the second inner chamber.

12. The system according to claim 5, wherein the predetermined amount of the aqueous liquid phase channeled as the aqueous liquid phase sample from the first inner chamber to the second inner chamber is substantially in the range of 50-60 milliliters.

13. A method for separating and in-situ analyzing a discrete sample of multiphase fluid, the method comprising:
introducing a discrete sample of multiphase fluid into a first inner chamber of a separation vessel, wherein an analytical cell having a second inner chamber is built-in inside the first inner chamber of the separation vessel, and wherein the analytical cell has a sample inlet for fluidly communicating the second inner chamber with the first inner chamber;
mixing a predetermined amount of demulsifier obtained from a demulsifier source with the discrete sample of multiphase fluid in the first inner chamber to cause the discrete sample to separate into liquid phases including an aqueous liquid phase and a nonpolar liquid phase;
channeling a predetermined amount of the separate aqueous liquid phase as an aqueous liquid phase sample from the first inner chamber to the second inner chamber via the sample inlet of the analytical cell, in response to determining that the discrete sample of multiphase fluid in the first inner chamber has separated into liquid phases including the aqueous liquid phase and the nonpolar liquid phase;
diluting the aqueous liquid phase sample channeled into the second inner chamber with a predetermined amount of fresh water from a fresh water reservoir to generate a diluted aqueous liquid phase sample; and
in-situ analyzing the diluted aqueous liquid phase sample contained in the second inner chamber with at least one probe having a sensing area disposed in the second inner chamber, wherein the second inner chamber is defined inside the first inner chamber.

14. The method according to claim 13, further comprising:
obtaining diluted aqueous liquid phase sample data based on the in-situ analysis with the at least one probe;
calculating nondiluted aqueous liquid phase sample data based on the diluted aqueous liquid phase sample data, as well as based on the predetermined amount of fresh water in the diluted aqueous liquid phase sample; and
transmitting the nondiluted aqueous liquid phase sample data to a multiphase flow meter.

15. The method according to claim 14, wherein the analytical cell further has a sample outlet on a bottom surface thereof, wherein the separation vessel has a drain outlet on a bottom surface thereof, and wherein the method further comprises:
draining the diluted aqueous liquid phase sample in the second inner chamber via the sample outlet after obtaining the diluted aqueous liquid phase sample data;
rinsing the second inner chamber and the sensing area of the at least one probe disposed in the second inner chamber with fresh water from the fresh water reservoir after draining the diluted aqueous liquid phase sample; and
draining the discrete sample of multiphase fluid in the first inner chamber via the drain outlet after channeling the predetermined amount of the aqueous liquid phase as the aqueous liquid phase sample from the first inner chamber to the second inner chamber.

16. A water analysis unit of a system for separating and in-situ analyzing a discrete sample of multiphase fluid, the water analysis unit comprising:
an analytical cell disposed inside a first inner chamber of a separation vessel for separating a discrete sample of multiphase fluid into liquid phases including an aqueous liquid phase and a nonporous liquid phase, wherein the analytical cell has: (i) a second inner chamber that is defined inside the first inner chamber, and (ii) a sample inlet to fluidly communicate the second inner chamber with the first inner chamber; and
at least one probe having a sensing area disposed in the second inner chamber for in-situ analysis of a sample of the aqueous liquid phase that is separated from the discrete sample of multiphase fluid in the first inner chamber and that is channeled to the second inner chamber from the first inner chamber for the in-situ analysis.

17. The water analysis unit according to claim 16, wherein the at least one probe has an oblong shape, and wherein the sensing area of the probe is covered with an ion-exchange membrane to prevent fouling of the sensing area.

18. The water analysis unit according to claim 16, wherein the analytical cell is built-in in a bottom portion of the separation vessel, and wherein an opening of the sample control valve is adapted to be disposed in a region of the first inner chamber where the aqueous liquid phase accumulates after separation thereof the discrete sample of multiphase fluid.

19. The water analysis unit according to claim 16, wherein the analytical cell further has a fresh water inlet, and the second inner chamber is in fluid communication with an external fresh water reservoir via the fresh water inlet, and wherein the water analysis unit further includes:
a sample control valve coupled to the sample inlet for controlling a flow of the aqueous liquid phase sample from the first inner chamber to the second inner chamber; and
one or more processors operatively coupled to the sample control valve and the at least one probe, the one or more processors being configured to:
control the sample control valve to allow a predetermined amount of the separate aqueous liquid phase to flow into the second inner chamber via the sample inlet as the aqueous liquid phase sample;
dilute the aqueous liquid phase sample in the second inner chamber to generate a diluted aqueous liquid phase sample by allowing a predetermined amount of fresh water from the fresh water reservoir to flow into the second inner chamber via the fresh water inlet;
in-situ analyze the diluted aqueous liquid phase sample in the second inner chamber with the at least one probe to obtain diluted aqueous liquid phase sample data;
calculate nondiluted aqueous liquid phase sample data based on the diluted aqueous liquid phase sample data, and based on the predetermined amount of fresh water in the diluted aqueous liquid phase sample;
transmit the nondiluted aqueous liquid phase sample data to an external multiphase flow meter.

20. The water analysis unit according to claim 19, wherein the sensing area of the at least one probe is at a distal end of the probe, and wherein the probe is oriented in the second inner chamber such that the sensing area is immersed in the diluted aqueous liquid phase sample when the diluted aqueous liquid phase sample is contained in the second inner chamber.

21. The water analysis unit according to claim 19, wherein the at least one probe includes an ion-selective electrode configured to in-situ measure one or more properties of the diluted aqueous liquid phase sample, the one or more properties selected from a group including: sodium concentration, chloride concentration, total dissolved solids (TDS) concentration, pH, conductivity, sulfate concentration, carbonate concentration, and nitrate concentration.

22. The water analysis unit according to claim 19, wherein the at least one probe includes first, second, and third probes that are proximally disposed adjacent to each other such that each probe is oriented in the second inner chamber with the sensing area of the probe in a downward direction and immersed in the diluted aqueous liquid phase sample when the diluted aqueous liquid phase sample is contained in the second inner chamber, and such that there exists an acute angle measured from the probe to a horizontal plane that is substantially perpendicular to a direction of gravity.

23. The water analysis unit according to claim 19, wherein the analytical cell further has a sample outlet on a bottom surface thereof, and wherein the one or more processors are further configured to:
drain the diluted aqueous liquid phase sample in the second inner chamber via the sample outlet after obtaining the diluted aqueous liquid phase sample data; and
rinse the second inner chamber and the sensing area of the at least one probe disposed in the second inner chamber with fresh water from the fresh water reservoir after draining the diluted aqueous liquid phase sample.

24. The water analysis unit according to claim 19, wherein the predetermined amount of the aqueous liquid phase allowed to flow into the second inner chamber via the sample inlet as the aqueous liquid phase sample is substantially in the range of 50-60 milliliters.
